SEQUENCE LISTING (12 sequences)

SEQ ID NO: 1 - RNA, 25 nt, human
ugcucaacgacaucgugauucucca

SEQ ID NO: 2 - RNA, 25 nt, Artificial Sequence
  antisense to Neutrophil elastase
uggagaaucacgaugucguugagca

SEQ ID NO: 3 - RNA, 25 nt, human
acgacaucgugauucuccagcucaa

SEQ ID NO: 4 - RNA, 25 nt, Artificial Sequence
  Neutrophil elastase set #2 antisense
uugagcuggagaaucacgaugucgu

SEQ ID NO: 5 - RNA, 25 nt, human
gcacaguuuguaaacuggaucgacu

SEQ ID NO: 6 - RNA, 25 nt, Artificial Sequence
  Neutrophil elastase set #3 antisense
agucgauccaguuuacaaacugugc

SEQ ID NO: 7 - RNA, 21 nt, Artificial Sequence
  negative control siRNA sequence Cy3
aucggugcgcuugucgcaguc

SEQ ID NO: 8 - RNA, 21 nt, Artificial Sequence
  unlabeled negative control siRNA sequence
gacugcgacaagcgcaccgau

SEQ ID NO: 9 - DNA, 24 bp, Artificial Sequence
  forward RT-PCR primer for ELA2
aatccacggaattgcctccttcgt

SEQ ID NO: 10 - DNA, 24 bp, Artificial Sequence
  antisense orientation RT-PCR primer for ELA2
ttgtcctcggagcgttggatgata

SEQ ID NO: 11 - DNA, 24 bp, Artificial Sequence
  sense (forward) RT-PCR primer for GAPDH
ttcgacagtcagccgcatcttctt

SEQ ID NO: 12 - DNA, 25 bp, Artificial Sequence
  reverse (antisense) rq-PCR primer for GAPDH
gcccaataccgaccaaatccgttga

(19) United States
(12) Patent Application Publication
(10) Pub. No.: US 2010/0151573 A1
(43) Pub. Date: Jun. 17, 2010

(54) Compositions and methods for delivery of molecules to selectin-ligand-expressing and selectin-expressing cells

(76) Inventors: Michael R. King, Ithaca, NY (US); Zhong Huang, Oklahoma City, OK (US)

Correspondence Address: HODGSON RUSS LLP;THE GUARANTY BUILDING, 140 PEARL STREET, SUITE 100, BUFFALO, NY, 14202-4040, US

(21) Appl. No.: 12/590,965

(22) Filed: Nov. 17, 2009

Related U.S. Application Data
(60) Provisional application No. 61/115,159, filed on Nov. 17, 2008.

(51) Int. Cl. C12N 5/00; C12M 1/00

(52) U.S. Cl. 435/375; 435/283.1

(57) ABSTRACT

The present invention is directed to methods for delivery of payload molecules to selected cells. The method comprises payload carrying delivery vehicles tagged with selectin or selectin-ligands. The payload carrying delivery vehicles are immobilized on flow surfaces and payload is delivered to targeted cells during rolling. The invention is also directed to compositions and devices for carrying out the method.

### CROSS-REFERENCE TO RELATED APPLICATIONS

This application claims priority to U.S. provisional patent application Ser. No. 61/115,159, filed on Nov. 17, 2008, the disclosure of which is incorporated herein by reference.

### BACKGROUND OF THE INVENTION

1. Field of the Invention

The present invention is directed to the targeting of various molecules and multi-molecular structures to selectin-ligand-expressing or selectin-expressing cells using selectin ligand-selectin interactions, to methods for accomplishing this targeting, to compositions useful for such methods, and to devices for use in these methods. Methods for targeting selectin-ligand-expressing or selectin-expressing cells with a multi-molecular structure comprising a delivery vehicle are particularly contemplated.

2. Description of Related Art

In recent years, studies have revealed that cells moving through the bloodstream (generically, “circulating cells”) do not passively flow past the endothelial cells lining the walls of the blood vessels (synonymously, “endothelial wall cells”). Instead, circulating cells expressing the appropriate surface molecules undergo rolling interactions with molecules that the endothelial wall cells express or are induced to express by a variety of signaling mechanisms, e.g., in response to inflammation in tissue near the vascular wall. Given the appropriate interaction(s), these circulating cells tether to the vessel wall, modify their cytoskeletal structures, and secrete protease enzymes that allow them to penetrate through the wall (“extravasate”) and migrate away from the point of penetration to targeted geographical areas within the body, e.g., a point of inflammation. See, e.g., Reinhart-King et al., Biophys. J., 89:676-689 (2005).

The class of molecules involved in cell rolling and rolling adhesion is the selectins, specifically the family of L-, P-, and E-selectin cell adhesion molecules (CAMs) which interact with various carbohydrate ligands (synonymously, “selectin ligands”) to cause circulating cell rolling and rolling adhesion. In terms of the expression of these molecules, L-selectin is expressed in circulating primitive hematopoietic stem and progenitor cells (“HSPCs”) as well as mature white blood cells (i.e., leukocytes, a group which includes the lymphocytes in which L-selectin expression was first observed; see. e.g., Sackstein, J. Invest. Derm., 122:1061-1069 (2004)). P-selectin, on the other hand, is expressed in circulating cells (specifically platelets) and additionally in endothelial wall cells, while E-selectin is expressed in endothelial wall cells only. In terms of the ligands that interact with these selectins, although the list is not complete, L-selectin interacts with CD34, sgp200, endomucin, GlyCAM-1, and MAdCAM-1 (see, e.g., Ley, J. Exp. Med, 198:1285-1288 (2003); E-selectin interacts with E-selectin Ligand-1 (ESL-1) and P-selectin glycoprotein ligand-1 (PSGL-1); and, P-selectin interacts with PSGL-1. It should be noted that selectin ligands all have the common feature of the sialylated-fucosylated carbohydrate sialyl Lewis^{x} (“sLe^{x}”) (see, e.g., U.S. Pat. Nos. 5,985,852 and 6,133,239, the contents of which are herein incorporated by reference).

Abundant evidence has demonstrated that these selectin ligand-selectin rolling interactions play an important role in many normal and disease processes. However, no attempts have been made heretofore to advantageously use the natural cell rolling phenomenon for targeting, capture and delivery of payload molecules to circulating cells. Currently utilized means for delivering such molecules into cells include liposomes, which are lipid-based vehicles. Liposomes are made of similar material to the cellular membrane, and so they have biocompatibility and biodegradability with living cells. They are formed by self-closing with one or more concentric lipid bilayers and an inner aqueous phase, in which the delivery molecules can be easily encapsulated and isolated from the surrounding environment. However, selective delivery of payload molecules to desired cells has been a challenge and release of liposomes into the circulation is often undesirable. Consequently, there continues to be an unmet need to develop methods and compositions for selective delivery of payload molecules to desired cells, particularly to circulating cells.

### BRIEF SUMMARY OF THE INVENTION

The present invention provides compositions, methods and devices for delivery of payload molecules to selected cells based on the selectin-selectin-ligand mediated cell rolling. In particular, this invention provides an effective method for delivering payload molecules (including but not limited to nucleic acids) to the interior of cells rolling over a selectin-coated or a selectin ligand-coated surface (such as a microtube through which blood flows). Payload carrying delivery vehicles tagged with selectin (or selectin-ligand) are immobilized on the inner surface of a flow channel (such as a microtube) and deliver the payload to targeted cells expressing a selectin ligand (or a selectin) during rolling of target cells.

The direct use of selectin ligand-selectin interactions offers up a variety of possible advantages over more conventional methods such as antibody labeling. For example, selectin ligand-selectin interactions on living cells are highly regulated interactions which may facilitate the delivery of particular molecules or molecular-structures to the target cells. As another example of the likely benefit of using such selectin ligand-selectin interactions for targeting, interactions such as antibody-antigen binding may disrupt or otherwise alter normal cell function in ways that may not occur for the selectin ligand-selectin interactions that the cells are evolved to undergo.

The present invention is directed to the targeting of various molecules and multi-molecular structures to selectin-ligand expressing or selectin-expressing cells using selectin ligand-selectin interactions, to methods for accomplishing this targeting, to compositions useful for such methods, and to devices for use in these methods.

In one non-limiting aspect, the present invention is drawn to the targeting of cells expressing ligands to P-selectin via hybrid P-selectin-lipid compositions. One specific embodiment of this aspect of the invention is the use of the P-selectin lipid hybrid molecule P-Selectin-disteroyl phosphatidylethanolamine-polyethyleneglycol (PS-DSPE-PEG) to form vesicular multi-molecular structures capable of delivering a payload such as RNA (including but not limited to siRNA) or DNA to cells, e.g. P-selectin-ligand-expressing cells. More specifically in this embodiment, recombinant human P-selectin (or a ligand binding portion thereof) is covalently attached to a lipid group (DSPE-PEG Maleimide) which allows the molecule to insert itself into the outer surface of lipid vesicles. siRNA is mixed with thin lipid film to form multilamellar lipid nanoparticles that encapsulate the siRNA (FIG. 2). These multilamellar vesicles are extruded to form unilamellar nanoparticles of diameter 100 nm (or in a range of 50 nm to 200 nm and all integers between 50 and 200) that are then reacted with the DSPE:P-selectin molecule to form lipid nanoparticles that contain siRNA (or other nucleic acid) and are coated with the adhesive P-selectin protein. Embodiments of this aspect of the invention in which the vesicles are surface-based and solution based are provided in Example 1 and prophetic Example 2 respectively. For example, a suspension of the nanoparticle construct may be incubated with a surface (e.g. a plastic microtube, an implant, or other structure) to produce a coating (FIG. 3). When a cell suspension is then perfused through this coated microtube, cells which express adhesive ligands to P-selectin (such as leukocytes or hematopoietic stem cells or metastatic cancer cells) slowly and adhesively roll across the tube surface. While the cells roll in close transient contact with the surface coating, the lipid nanoparticles fuse with the cell membrane and the siRNA (or other nucleic acid) enters into the cell (FIG. 4). This method can be generalized to other adhesion molecules other than P-selectin. For example, prophetic Example 3 is provided, in which cells expressing the E-selectin ligand HCELL (i.e. hematopoietic stem and progenitor cells, or “HSECs”) can be targeted with delivery vehicles comprising E-selectin-lipid hybrid molecules, particularly such delivery vehicles further comprising payloads such as an siRNA payload (or other genetic material). In one embodiment, a mixture of adhesion molecules can be used to tune the selectivity to pull rare cells (e.g. circulating tumor cells, mobilized stem cells) out of the blood stream (in vivo or ex vivo) or from a mixed cell population (e.g. in vitro). siRNA probes can be designed to knock down (e.g. silence) any gene of interest. For example, the elastase-2 gene can be knocked down in neutrophil precursor cells to treat rheumatoid arthritis. Alternatively, the selectin coated nanoparticles can encapsulate DNA (e.g. a plasmid or vector containing any gene) that one wishes to insert into the cell's genome, even genes of non-human origin.

Another embodiment of this invention is to dispense with the microtube altogether and to take P-selectin-coated nanoparticles containing siRNA or DNA and mix these directly with a cell mixture such as blood (in vivo or ex vivo). The nanoparticles will directly bind to cells which possess P-selectin ligand and become taken up inside the cell, while the precious siRNA will not be wasted on the majority of non-targeted cells.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee. The following drawings are provided in order to provide further description of the present invention. These drawings are not intended to limit the present invention in any way.

FIGS. 1a-c. Generation of P-selectin Receptor Specific Nanoparticles. (a) Traut's Reagent (2-Iminothiolane HCl) reacts with primary amines (—NH₂) of P-selectin, which introduces sulfhydryl (—SH) groups to the protein. The sulfhydryl group of P-selectin reacts with the maleimide group of DSPE-PEG2000 maleimide to form a DSPE-PEG-P-selectin conjugate. R¹ represents P-selectin, R² represents DSPE-PEG(2000) Maleimide. (b) Multilamellar particles were formed by hydration of lipid thin films with protamine and thymus DNA packaged siRNA and extruded to produce unilamellar nanoparticles (UNP). P-Selectin-DSPE-PEG or DSPE-PEG were inserted into UNP to form P-selectin receptor specific nanoparticles or non-targeting nanoparticles, respectively. (c) Scanning electron microscopy of liposomes. Scale bars indicate the micrograph magnifications.

FIG. 2. P-selectin is necessary for the PS-DSPE-PEG-nanoparticles to absorb onto the surface of microtubing. (a) The microtubing was coated with PS for 2 h and then coated with nanoparticles for another 2 h. (c) The microtubing was coated with PS-DSPE-PEG nanoparticles for 2 h. The pictures of (a) and (c) were taken before perfusion. The pictures of (b) and (d) were taken after perfusion and before the cells were released.

FIGS. 3a-b. siRNA uptake and gene knockdown efficiencies in different lipid vesicles. (a) Fluorescence intensities of cell lysate from cells treated with different Cy3-siRNA lipid vesicles. HL60 cells were incubated with different Cy3-siRNA lipid vesicles at 37° C. for 4 h. Cells were washed and lysed and cell lysate analyzed for fluorescence intensity using a fluorescence spectrometer (absorbance 550 nm; emission 564 nm). Results are expressed as mean±SD (n=3). ** represents p<0.01. “Cy-3 siRNA Liposome” indicates unilamellar liposome; “DSPE-PEG NP” is unilamellar liposome with attached DSPE-PEG; “PS-DSPE-PEG NP” is unilamellar liposome with PS-DSPE-PEG attached. Panel b show the knockdown levels of ELA2 mRNA rq-PCR. * represents p<0.05.

FIGS. 4a-b. P-selectin and its receptor are required for siRNA-nanoparticle uptake and target gene knockdown. (a) Different liposomes were taken up by HL60. The & symbol represents HL60 cells pre-incubated with anti-PSGL-1, and && represents PS-DSPE-PEG-nanoparticles pre-incubated with anti-P-selectin before the uptake experiments. Data are presented as the mean of three experiments. ** represents p<0.01, where fluorescence intensities of the PS-DSPE-PEG-nanoparticle group were compared to the other groups. (b) is from rq-PCR, showing knockdown levels of ELA2 gene using different ELA2 siRNA nanoparticles as indicated.

FIGS. 5a-v. P-selectin and its receptor are necessary for nanoparticles to absorb onto the surface of microtube and interact with HL60 cells. P-selectin nanoparticles were coated onto the surface of microrenathane tubing for 2 h at RT, and then perfusion experiments conducted. (a) and (b) were taken before perfusion, (c) and (d) were taken after perfusion but before the cells were released. (e) and (f) were taken after the adherent cells were released from the microtubing. In (g) and (h), HL60 cells were perfused through the P-selectin coated tube. In (i) and (j), cells were pre-incubated with anti-PSGL-1 for 1 h at RT and then perfused through the P-selectin coated tube. In (k) and (l), the perfused cells were MCF7. In (m) and (n), the P-selectin nanoparticles were incubated with anti-P-selectin for 1 h at RT and then applied to the microtube coating. In (o) and (p), P-selectin was replaced with IgG to construct the coating nanoparticles. The pictures were taken after cellular perfusion but before the cells were released from the surface. [III]. Nanoparticles alone could not (q, r, s) but P-selectin nanoparticles could (t, u, v) deliver Cy3-siRNA into HL60 cells under rolling conditions in the microtubing. q and t were taken in epifluorescence mode, r and u were taken in brightfield mode, and s and v are the merge of q and r, and s and t respectively. TRITC represents Tetramethyl Rhodamine Iso-Thiocyanate filter.

FIGS. 6a-d. The neutrophil elastase gene was knocked down by siRNA-PS-DSPE-PEG-nanoparticles under rolling conditions in microtube. The transcriptional level of ELA2 was quantified by rq-PCR as shown in bar graph form (6a). The data from rq-PCR are normalized with GADPH. Data are presented as mean±SD (n=4). Significant differences are indicated by asterisks, * represents p<0.05, and ** represents p<0.01. The Western blots (6b) were probed with an antibody against ELA2 (c upper panel) and GAPDH (b lower panel). Progress curves show the relative activity of human neutrophil elastase (6c) in control and three sets of neutrophil elastase siRNA transfected in HL60 cells under rolling conditions in microtubing. Data are presented as the mean of three experiments with 95% confidence intervals. * represents p<0.05. The mRNA level of ELA2 was quantified by rq-PCR (6d). 1 represents HL60 cells that were transfected with control and ELA2 siRNAs under static conditions. 2 represents the HL60 cells that were transfected with control and ELA2 siRNAs under perfusion conditions. The data from rq-PCR are normalized with GADPH. Data are presented as mean±SD (n=4 in each group). Significant differences are indicated by asterisks, * represents p<0.05, and ** represents p<0.01.

FIGS. 7a-e. Differentiated HL60 cells can be transfected, and ELA2 gene can be silenced under rolling conditions by siRNA-PS-DSPE-PEG-nanoparticles. HL60 cells were induced into granulocytes by DMSO. After differentiation, the cells were perfused through the tube coated with PS-DSPE-PEG-nanoparticles. The picture (a) and (b) were taken after cell perfusion but before the cells were released from the tubes. The pictures of (c), (d) and (e) were taken after the adherent cells were collected from the tubes and cultured for 36 hours.

FIGS. 8a-b. The levels of gene knockdown are shown by rq-PCR (8a) and Western blot (8b).

### DETAILED DESCRIPTION OF THE INVENTION

The present invention provides compositions, methods and devices for selective delivery of molecules to cells. Payload carrying delivery vehicles (nanoparticles) are tagged with selectin (or selectin-ligand) and are immobilized on to the inner surface of flow channels. Selective delivery of the payload molecules to targeted cells flowing through the flow channels advantageously uses the rolling of cells via selectin selectin-ligand interactions.

### 1. Selectin Ligand—Selectin Targeting Generally.

The present invention is directed to the use of selectin ligand-selectin interactions to target molecules and multi-molecular structures such as vesicles or other delivery vehicles to selectin-expressing cells or selectin-ligand-expressing cells. The invention is directed to methods for accomplishing such targeting, to compositions used in such methods, and to devices for practicing these methods.

Accordingly, in the present invention, cell rolling has been successfully applied to tether and capture specific target cells on the surface of microfluidic channels by interaction with endothelial cell adhesion molecules immobilized on to the surface of the channels. Desired molecules are delivered to the cells during the process of cell rolling. To achieve specific targeting, surface modification of delivery vehicle nanoparticles (such as liposomes) is carried out. Surface modification endows the nanoparticles not only with target specificity but also with the ability to adhere onto the inner surface of microtubes. Derivatization with polyethylene glycol is used to enhance the stability of the payload carrying nanoparticles. In addition to particle stabilization, PEGylation of the nanoparticles also neutralizes the positive charge of the nanoparticles. It is believed that stabilization enhances the ability of the particles to resist elution under shear stress.

Thus, in one embodiment, this invention provides a method for delivering a payload to a cell expressing a cell surface selectin ligand comprising providing liposomes which have selectin molecules covalently attached to lipid molecules incorporated in the liposomal membrane. Payload molecules are encapsulated in the liposomes. The liposomes are allowed to attach to a flow surface (such as the inner surface of a fluidic channel e.g., a microtube) thereby providing a flow surface having selectin molecules thereon. Then a fluid is allowed to flow in the surface modified fluidic channel under conditions permitting rolling of cells over said flow surface. This results in delivery of the payload molecules to the cells. By “immobilized” as used herein, is meant that the liposomes are attached, such as by non-specific physisorption, on a flow surface such that the liposomes remain attached to the flow surface under flow conditions such as during flow of fluids comprising cells. An example of a flow of fluid includes the circulation of blood in the various blood vessels. For example, the liposomes should preferably remain immobilized at physiological wall shear stresses of 0.5 to 10 dyne/cm², and should preferably only detach when bound to rolling cells.

“Molecule” and “cell” as used herein are intended to refer to a particular type of molecule or cell rather than a single molecule or a single cell. As is described extensively herein, the present invention is directed to both the use of single molecule types and single cell types and, when appropriate, the use of multiple molecule types and multiple cell types. Thus Example 1 provides a P-selectin containing delivery vehicle that comprises multiple molecule types (P-selectin-lipid hybrid, lipids, Cy3-siRNA, etc.), while applications of the present invention to circulating cell populations would include multiple cell types.

“Targeting” as used herein refers to incubating or otherwise exposing a selectin-ligand containing molecule or multi-molecular structure to a cell expressing at least one type of selectin under conditions where selectin ligand-selectin interactions occur, an endpoint that may be functionally defined (e.g., by successful binding, delivery of targeted molecule(s), etc.). “Targeting” also includes the situation complementary to that just described, in which the molecules or multi-molecular structures comprise selectin and the cells express at least one type of selectin ligand.

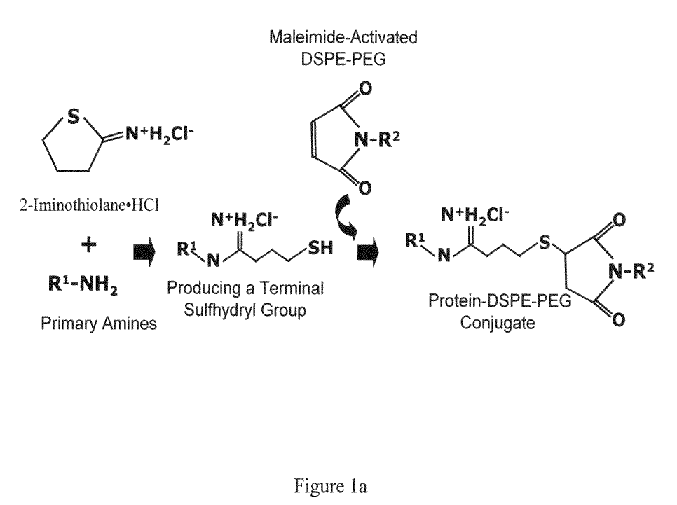

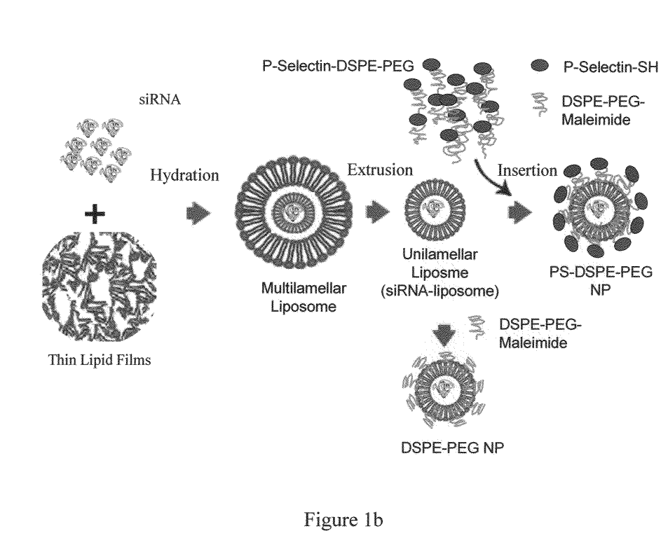

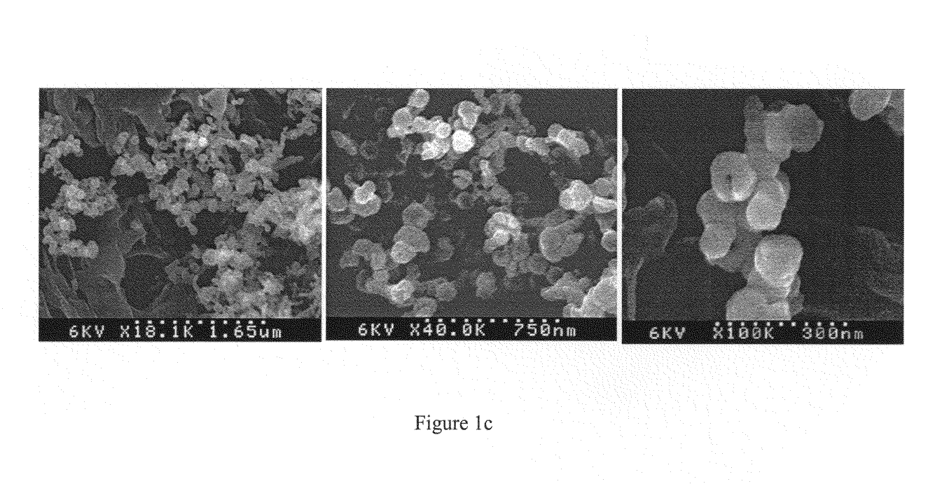

Thus, one aspect of the present invention is directed to targeting molecules and multi-molecular structures to cells expressing a particular selectin ligand. Leukocytes, for example, express the selectin ligand PSGL-1. Thus in this non-limiting example molecules and multi-molecular structures can be targeted to leukocytes via P- or E-selectin, which can be directly attached to the molecules and structures of interest (see, e.g., the hybrid P-selectin-lipid of FIG. 1) or otherwise associated with these molecules and structures (see, e.g., the PS-DSPE-PEG NPs of FIG. 2) so as to bring them into contact with the targeted cells.

The previous paragraph describe targeting of cells expressing a selectin ligand, however, the present invention also includes targeting of molecules and larger structures to cells expressing a selectin. L-selectin, for example, is expressed on leukocytes; therefore, an L-selectin ligand such as PSGL-1 may be used to target molecules or multi-molecular structures directly attached to PSGL-1 or associated with the PSGL-1 to these L-selectin expressing cells. Other cancer cell selectin ligands are more selective, for instance CD44v which binds to E-selectin but not P-selectin.

As noted above, the present invention depends upon the existence of one or more selectin ligand-selectin interactions. Although various embodiments of the invention described herein discuss situations in which only a single such selectin ligand-selectin interaction is utilized for targeting, this language should not be interpreted as limiting; instead, the present invention explicitly contemplates both situations in which there is only one such interaction that is utilized and situations in which more than one such interaction is utilized.

In this regard, Applicants note that in some situations a cell may normally exhibit the capacity for only a single such interaction, i.e., the cell expresses only a single selectin ligand or only a single selectin in its natural state and biological context. In such situations it may be preferable to use only this single mechanism for targeting. However, it may also be possible to induce the expression of additional selectin ligands or selectins on the desired cells by environmental cues or by recombinant methods (or both), and such situations are explicitly contemplated in the present invention.

With regard to the choice of a particular selectin ligand-selectin interaction when multiple such interactions are available or can be engineered, Applicants note that the choice of which interaction or interactions will be most advantageous will depend upon a variety of factors including especially the selectivity that the choice of a particular such interaction or interactions confers. As already noted, for example, it appears that the E-selectin ligand HCELL is expressed only on hematopoietic stem and progenitor cells (“HSECs”), while L-selectin is expressed on HSECs and also on mature white blood cells, e.g. leukocytes. Therefore given a population of circulating cells, selectivity for HSECs will be highest when E-selectin is used for targeting rather than when an L-selectin ligand such as PSGL-1 is used. Therefore, given this situation, E-selectin may be the best choice for targeting. Applicants note that this logic is explicitly part of the determination process for the best selectin ligand-selectin interaction for use in the present invention.

Although the preceding discussion has been directed to the use of a selectin ligand-selectin interaction or interactions to obtain cell targeting, the invention explicitly includes cell targeting in which additional interactions are included in order to more finely tune the interaction(s), specificity, etc.

For example integrins may also be included as interacting molecules, as may other adhesion molecules. Appropriately chosen antibodies may also enhance the properties of selectin ligand-selectin interactions, particularly in light of previous observations of the additional effects of selectins co-immobilized with antibodies on flowing antigen-coated beads (Eniola et al., Biophys. J., 85:2720-2731 (2003); see also the discussion of this topic in Charles et al., Biotechnol. Prog., 23:1463-1472 (2007)). A convenient functional assay for the selection of the appropriate number and types of interactions is the successfulness of targeting, specificity, etc.

### 2. Contemplated Selectin Ligands and Selectins.

The selectin ligands and selectins that may be used in the present invention includes all ligands currently identified as binding selectins, and all molecules currently identified as selectins including, without limitation, naturally occurring and modified selectin ligands and selectins and mutants thereof, whether modification is by recombinant DNA technology, chemical modification, etc. In one embodiment, the modified selectin may be a selectin-Fc chimera, however, any other chimeras or mutants may also be used.

With regard to modifications of these molecules, Example 1 provides an example of one such category of modification that has particular utility, i.e., a selectin that is modified by attachment to a lipid group such that the hybrid selectin-lipid will be incorporated into a lipid bilayer. Specifically, Example 1 provides a DSPE-PEG-P-selectin hybrid (see also FIG. 1 and accompanying legend); this example is not limiting, and other such hybrid molecules are also contemplated.

### 3. Molecules, Multi-Molecular Structures, and Delivery Vehicles.

The present invention is broadly directed to the targeting of molecules and larger multi-molecular structures to cells via selectin ligand-selectin interactions. At a minimum, such interactions will bring either a selectin ligand or a selectin in proximity to the targeted cell. Such a situation will, by itself, have utility by, e.g., blocking cellular selectin sites or cellular selectin ligand sites, thereby interfering with processes requiring such sites, e.g., cell rolling.

However, although the present invention includes such unmodified selectin ligands and unmodified selectins, in one embodiment, the invention is directed to situations in which these molecules are modified to provide enhanced utility, e.g. are modified to deliver a desired probe, functionality, activity, or delivery vehicle.

Thus, for example, the delivered molecule may be tagged with a probe that allows for visualization of the delivered molecule, or an enzymatic activity, etc. Additionally, and as provided in, e.g., Example 1, the selectin ligand or selectin may be modified so as to bring a delivery vehicle into proximity with the targeted cell.

As used herein, “delivery vehicle” refers to any of the various structures developed to envelop, encapsulate, incorporate or otherwise package for delivery a material that is to be brought into proximity with a target. Such delivery vehicles include, but are not limited to, liposomes, vesicles, polymerosomes, etc. A delivery vehicle will of necessity comprise at least one type of selectin molecule or selectin-containing molecule when the delivery vehicle is to be targeted to selectin ligand-expressing cells; conversely, the delivery vehicle will comprise at least one type of selectin ligand molecule or selectin-ligand containing molecule when targeting is to a selectin-expressing cell. However, it is understood that a delivery vehicle may also include other molecules than a type of selectin or type of selectin-ligand. Accordingly, in one embodiment, the delivery vehicles of the present invention can be conferred further selectivity based on additional affinity binding pairs. For example, the delivery vehicles can have, in addition to selectin (or selectin ligand), covalently attached antibodies to surface antigens found on specific cells (such as specific cancer cells). As another example the delivery vehicle can additionally have covalently attached extracellular matrix proteins or the RGD peptide sequences which are known to bind to epithelial cells via integrin:ligand interactions. The binding of epithelial type cells is particularly desirable for targeting rare circulating tumor cells in blood.

In one particularly preferred aspect of the invention, the delivery vehicle is formed as a result of covalent attachment of a selectin molecule to a lipid group such that the hybrid molecule can associate with/assemble into a vesicle; a non-limiting example of such a hybrid molecule constructed with P-selectin is provided in Example 1. As shown in this Example and particularly in FIGS. 1-2, such hybrid molecules will assemble into vesicles to form P-selectin-decorated vesicles; “decorated” refers to the surface presentation of (in this case) P-selectin, and represents a statement of functionality. That is, “decoration” refers to a physical structure that results in the decorating molecule or molecular group being presented in such a way that it is able to interact with other molecules. Thus, “decoration” with P-selectin is taken to mean that the P-selectin is presented in such a way that it can interact with its binding ligand(s).

Another example of the attachment of a selectin to a lipid group is provided in Example 3, in which E-selectin can be attached to a lipid group for assembly into a delivery vehicle similar to that of Examples 1-2.

In one embodiment, the delivery vehicle is a liposome. The liposome may be unilamellar or multilamellar. In one embodiment, the size of the liposomes is 50 to 200 nm and all integers between 50 and 200 nm in diameter. In one embodiment, the liposomes are 90 to 110 nm and in another embodiment, the liposomes are 100 nm. The liposomes comprise phosphoglycerides such as, but not limited to, phosphatidylcholine, phosphatidylserine, phosphatidylethanolamine and/or phosphatidylglycerol. The phosphoglycerides have two acyl chains. In one embodiment, the length of the acyl chains attached to the glycerol backbone can be from 12 to 22 carbon atoms. The acyl chains may be saturated or unsaturated. Some non-limiting examples of 12-22 carbon atom saturated and unsaturated acyl chains include lauric acid, myristic acid, palmitic acid, stearic acid, arachidic acid, behenic acid, oleic acid, palmitoleic acid, linoleic acid, and arachidonic acid. The chains of the phosphoglycerides may be of the same length or different length. Thus, in various embodiment, the liposomes comprise disteroyl phosphatidyl choline (DSPC); dimyristoyl phosphatidyl glycerol (DMPG); and/or disteroyl phosphatidylethanolamine (DSPE).

It is generally preferable to attach a polyethylene glycol (PEG) molecule to the phosphoglycerides. This helps in stabilization of the liposome. The selectin or the selectin ligand molecules can be covalently attached to PEG-phosphoglycerides by any of a variety of known methods in the art. For example, a chemically reactive end group such as hydrazide, N-(3′-(pyridyldithio) proprionate, maleimide, succinyl, p-nitrophenylcarbonyl or cyanuric chloride may be used.

If a selectin ligand (such as P-selectin glycoprotein ligand-1 (PSGL-1) or the small molecule tetrasaccharide sialyl Lewis x) is to be covalently attached to the delivery vehicles, then a covalent linkage can be established covalently via carboxyamine chemistry for example.

Additionally, selectin or selectin ligands can be covalently bound to the liposomes via the use of other linkages such as avidin-biotin pairs or his-tag:antibody.

Preparation of Liposomes is Well Known in the Art. One Having Skill in the Art will recognize the numerous liposome compositions and methods of producing liposomes. Typically, properties of lipid formulations can vary depending on the composition (cationic, anionic, neutral lipid species). Generally, the elements of the procedure for liposome preparation include preparation of the lipid for hydration, hydration with agitation, and sizing (e.g., sonication, extrusion, etc.) to a homogeneous distribution of vesicles. For example, liposomes can be prepared using a thin-film hydration protocol.

### 4. Payloads.

The delivery vehicles of the previous section are typically intended to carry a payload of at least one kind of agent, i.e., at least one visualization agent, enzyme, polymer, therapeutic agent (such as doxorubicin) etc., as would be routinely contemplated by one of ordinary skill in the art. Thus, in various embodiments, the payload molecules may be proteins, nucleic acids, carbohydrates or combinations thereof or any other biological molecules. In various embodiments, different payload molecules may be packaged into a single liposome or combinations of liposomes having different payloads may be used.

In one embodiment of the present invention, the payload is genetic material suitable for changing gene expression in the targeted cell, either by the introduction of new genetic material on a transient or permanent basis, or by the modulation of endogenous genetic material (e.g. by silencing RNA, or “siRNA.”). For targeted stem cells, for example, siRNA molecules or other constructs capable of altering gene expression (especially for programming/reprogramming) are particularly contemplated.

### 5. Devices

The present invention is also directed to devices/apparatus that may be used to carry out the methods of the invention, whether in vitro or in vivo. In this regard Applicants explicitly contemplate devices such as those described in U.S. Patent Publ. Nos. 2006/0183223 and 2007/0178084, the contents of which are incorporated herein.

Although the preceding discussion has been directed to the use of a selectin ligand-selectin interaction or interactions to obtain cell targeting, the invention explicitly includes cell targeting in which additional interactions are included in order to more finely tune the interaction(s), specificity, etc.

In one embodiment, the device comprises one or more fluid flow channels, with each channel comprising a flow surface. The flow channels are preferably made of a material that allows immobilization of the delivery vehicles (such as liposomes). The delivery vehicles preferably remain immobilized under shear forces such as those encountered during flow of fluids through these channels during targeting and capture of cells, including shear forces experienced under physiological conditions. Those skilled in the art will recognize that the attachment of liposomes on a flow surface is affected by, for example, surface properties of the flow surface (which are in part determined by the material comprising flow surface), the surface properties of the liposomes, and the composition of the flow solution. It is considered that based on the teachings herein, determining the appropriate conditions necessary to achieve immobilization of liposomes on the flow surface is within the purview of one having skill in art. For example, various biocompatible polymers known in the art can be used as flow surfaces. In one embodiment, microrenathane flow channels can be used.

In one embodiment, the flow channels are microtubes having a diameter of from 20 to 1000 microns and all integers therebetween. This diameter range is similar to the physiological blood vessel diameters in which red blood cells migrate across streamlines to the center of the vessel, and displace leukocytes, and stem and cancer cells towards the walls, a process called “margination”. A device may have a single flow channel or may have multiple flow channels which may be parallel flow channels. In one embodiment, the flow channel has selectin tagged liposomes immobilized to the flow surface such the liposomes remain immobilized at wall shear stress of 0.5 to 10 dyne/cm² and all integers and values to the tenth decimal place between 0.5 to 10.

An advantage of the delivery vehicles being immobilized on the flow surfaces is that during use of the present device, unwanted release of the liposomes into the circulating fluid in minimized or eliminated. This would be particularly important during in vivo use of device implants.

### 6. Disease Applications of the Present Invention

The present invention is directed to the targeted delivery of molecules or multi-molecular structures based on selectin ligand-selectin interactions for a variety of utilities as already discussed. One particular utility of the present invention is for the treatment of disease states.

In this regard Applicants note that targeting of stem cells for, e.g., gene delivery or siRNA will have particular advantages for the treatment of human or animal diseases. For example, reductions in the expression of elastase-2 gene to neutrophil precursor cells via the targeted delivery of siRNA may be an effective means to treat rheumatoid arthritis. Other such applications involving targeting to stem cells (a term which is broadly intended to include pluripotent cells, totipotent cells, precursor cells, etc.) are explicitly contemplated herein. The present invention can be used to deliver payload molecules to targeted cells in any circulation including specific blood cells, circulating blood cancer cells and metastatic cells.

### EXAMPLE

### Materials and Methods

### Cell Lines and Culture.

HL60 and MCF7 cell lines were purchased from American Type Culture Collection (ATCC, Manassas, Va.), and maintained in RPMI 1640 and DMEM (Gibco-Invitrogen, Carlsbad, Calif.), respectively, and supplemented with 100 IU/ml penicillin, 10 μg/ml streptomycin and 10% heat-inactivated fetal bovine serum in 5% CO₂ at 37° C. The DMSO-induced differentiation of HL-60 cells into granulocytes was conducted by adding 1.5% (v/v) dimethyl sulfoxide (Sigma-Aldrich, St Louis, Mo.) into the growth medium for 7 days. The medium was changed every 2 days.

Preparation of siRNAs.

The human neutrophil elastase siRNAs were from Invitrogen (Carlsbad, Calif.). The negative control and Cy3-negative control siRNAs were purchased from Integrated DNA Technologies (Coralville, Iowa). The siRNAs purchased from Integrated DNA Technologies were annealed according to the manufacturer's instructions. Human neutrophil elastase siRNA sequences were as follows:

Neutrophil elastase set #1:

(SEQ ID NO.: 1)

5′- UGCUCAACGACAUCGUGAUUCUCCA -3′ (sense #1);

(SEQ ID NO.: 2)

5′- UGGAGAAUCACGAUGUCGUUGAGCA -3′ (antisense #1);
Neutrophil elastase set #2:

(SEQ ID NO.: 3)

5′- ACGACAUCGUGAUUCUCCAGCUCAA -3′ (sense #2);

(SEQ ID NO.: 4)

5′- UUGAGCUGGAGAAUCACGAUGUCGU-3′ (antisense #2);
Neutrophil elastase set #3:

(SEQ ID NO.: 5)

5′- GCACAGUUUGUAAACUGGAUCGACU -3′ (sense #3);

(SEQ ID NO.: 6)

5′- AGUCGAUCCAGUUUACAAACUGUGC-3′ (antisense #3);

The sequences of Cy3-negative control siRNAs and the sequences of the unlabeled negative control were the same, and are as follows:

| | |
|---|---|
| 5′- AUCGGUGCGCUUGUCGCAGUC -3′ | (SEQ ID NO.: 7) |
| 5′- GACUGCGACAAGCGCACCGAU -3′ | (SEQ ID NO.: 8) |

The condensation of siRNAs were prepared by mixture of 50 μl of 50 μM siRNAs with 20 μl of 2 mg/ml protamine, 16 μl of 2 mg/ml of double strand calf thymus DNA (Sigma-Aldrich, St. Louis, Mo.) and DNA RNAase nuclease free water (Gibco-Invitrogen, Carlsbad, Calif.) up to 200 μl, followed by incubation at RT for 10-15 min.

siRNA-Liposome Preparation.

Multilamellar liposomes (MLL), composed of 1,2-Dioleoyl-3-Trimethylammonium-Propane (DOTAP), 1,2-Dipalmitoyl-sn-Glycero-3-Phosphocholine (DPPC) and cholesterol (Chol) (Avanti Polar Lipids, Alabaster, Ala.) at molar ratios of 0.5:1.75:1.75 (DOTAP:DPP:Chol), were prepared by routine method. Briefly, a total of 380 nM of lipids were dissolved in chloroform in a glass tube and gently dried under nitrogen and further evaporated to dryness under vacuum. The lipid film was hydrated with a swelling solution composed of protamine and thymus double strand DNA-condensed siRNA (2.5 nmol) dissolved in DNA and RNAase nuclease free water to create MLL at a total lipid concentration of 1.9 mM. The resulting MLL were sized by repeated thawing and freezing, and then subjected to extrusion (Avanti Polar Lipids, Alabaster, Ala.) through polycarbonate membranes (Nucleopore, Whatman, N.J.) with gradually decreasing pore size (0.4, 0.2 and 0.1 μm) to produce unilamellar nanoscale liposomes (ULNL) with 30 cycles each at 50° C. (FIG. 1b). The efficiency of siRNA entrapment was determined by a Quant-iT™ RiboGreen™ RNA assay (Molecular Probes, Invitrogen) according to the manufacturer's instruction. The intensity of siRNA encapsulated within liposomes was measured in the presence or absence of Triton X-100 at wavelengths of excitation 480 nm and emission 520 nm. The nanoparticles were freshly prepared and diluted with PBS, and the mean particle diameter and surface charge (zeta potential) measured by dynamic light scattering and Malvern Zetasizer nano ZS™ (Malvern Instruments Ltd. Worcestershire, UK), according to the manufacturers' protocols. The shapes and sizes of the ULNLs were also observed by scanning electron microscopy. Samples were rapidly frozen in liquid propane and immediately transferred to 1% osmium acetone (−196° C.). Samples were freeze-substituted over four days. SEM samples were then critical point dried, mounted and then sputter coated. Images were taken on a Hitachi 5900.

### Cellular Uptake Study

HL60 cells (1.5×10⁵ cells/0.5 mL well) were cultured in 24 well plates (Corning Inc., Corning, N.Y.) for 20 hours. Cells were treated with naked Cy3-siRNA and different Cy3-siRNA nanoparticles in the culture medium at 37° C. and 5% CO₂ for 4 hours. Cells were harvested by centrifugation and washed three times with PBS followed by incubation in 300 μL lysis buffer (1% Triton X-100 in PBS) at 4° C. while vortexing for 30 minutes. Fluorescence intensity of 200 μL of cell lysate was measured by a SpectraMax M2/M2e Microplate Reader (Molecular Device, Sunnyvale, Calif.) at wavelengths of excitation 550 nm and emission 570 nm.

### Preparation of Target Specific and Stabilized Nanoparticles.

Recombinant human P-selectin/Fc chimera (rhP/Fc) (R&D Systems, Minneapolis, Minn.) was dissolved in PBS and centrifuged with Microcon YM-30 30 kDa molecular weight cut-off filters (Millipore; Billerica, Mass.) to concentrate and remove salt from the protein. The resulting rhP/Fc was incubated with Traut's reagent (Pierce, Rockford, Ill.) in PBS at RT for 1.5 hours to introduce sulfhydryl (—SH) groups into the proteins (FIG. 1a). The molar ratio of rhP/Fc to Traut's Reagent was 1:7.5. The excess Traut's reagent was removed with a YM-30 column. The coupling reaction was carried out by mixing NHS-activated rhP/Fc with 1,2-Distearoyl-sn-Glycero-3-Phosphoethanolamine-N-Maleimide 2000 (DSPE-PEG2000 maleimide) (Avanti Polar Lipids, Alabaster, Ala.) at 4° C. overnight (FIG. 1a). The molar ratio of rhP/Fc to DSPE-PEG2000 maleimide was 1:3. The non-immune stimulated IgG1 (BD Pharmingen, Franklin Lakes, N.J. USA) as isotype-matched negative control was used to construct IgG-DSPE-PEG2000 maleimides as the procedure of rhP/Fc-DSPE-PEG2000 maleimide described above. Non-targeting and targeting nanoparticles were generated by incubating the mixture of 5 μL ULNL suspension with 15 μL micelle solution of DSPE-PEG2000 maleimide, IgG-DSPE-PEG2000 maleimides or rhP/Fc-DSPE-PEG2000 maleimide, respectively, at 50° C. for 15 min, and then cooled down at RT. The molar ratio of total lipid to DSPE-PEG2000 maleimide was 19:1 (FIG. 1b).

### Microtube Surface Preparation

For experiments with P-selectin targeting nanoparticles, 20 μL of nanoparticle solution was diluted with 130 μL of PBS and then perfused into blood-compatible microrenathane tubing (300 μm ID; Braintree Scientific, USA), and incubated for 2 h at RT under sterile conditions. For non-targeting nanoparticles, microtubing was pre-incubated with rhP/Fc at 60 μg/ml concentration in PBS for 2 hours. The non-absorbed rhP/Fc was removed by a gentle PBS wash, and then coated with non-targeting nanoparticles at the same concentration as P-selectin targeting nanoparticles for 2 h at RT. The nonspecific blocking of the lumen surface was achieved by 1 h incubation with 5% bovine serum albumin (BSA, Sigma-Aldrich, St. Louis, Mo., USA) dissolved in PBS. Following a gentle PBS wash, the absorbed P-selectin was activated in calcium-enriched Hanks balanced salt solution [2 mmol/L Ca⁺⁺] (HBSS, GIBCO) for 1 h at RT.

### Cell Capture and Collection

Cell capture and collection experiments were performed as follows. After surface coating, microtubes were positioned on an inverted epifluorescence microscope (IX-81; Olympus USA, New York, N.Y., USA) coupled to a CCD camera (Hitachi, Japan) for direct visualization of the adherent cells within the tube lumen. 2×10⁶/mL of HL60 or differentiated HL60 cells in HBSS enriched with calcium and 5% BSA were incubated at RT for 30 min and then perfused through the tube at a rate of 16 μL/min (wall shear stress 1 dyn/cm²) for 30 min and then at a rate of 32 μL/min (wall shear stress 2 dyn/cm²) for another 30 min, and then the adherent cells in the tubes were washed with HBSS enriched with calcium at a rate of 32 μL/min (wall shear stress 2 dyn/cm²) for 20 minutes and another 20 minutes at a rate of 48 μL/min (wall shear stress 3 dyn/cm²) using a motorized syringe pump system. The elusion of adherent cells was performed using a combination of high shear (flow rate 160 μL/min) and air embolism. The collected cells were cultured in 24 well dishes with 500 μL of growth medium. The transfection efficiencies were quantified by epifluorescence microscopy (Olympus IX81, Olympus America Inc) after 36 hours of culture.

Real-Time Quantitative PCR (rq-PCR)

The different nanoparticles carrying ELA2 or control siRNAs were transfected into HL60 cells either under flow or static conditions. The total RNA of the cells was harvested 48 hours after transfection. Total RNA was extracted by Trizol reagent, treated with DNase I, and first-strand cDNAs were synthesized with the SuperScript® First-Strand Synthesis System (Invitrogen, Carlsbad, Calif.) according to the manufacturer's instructions.

Real-time quantitative polymerase chain reaction (rq-PCR) was carried out with a Rotor-Gene 3000 real-time thermal cycler (Corbett Robotics, Australia) following the manufacturer's instructions. 20 μL reactions were set up using the cDNA transcribed from 2.5 ng of total RNA, 0.1 mM each of forward and reverse primer and the QPCR SYBR Green mix (Fisher Scientific, Fairlawn, N.J.). The following primers were used for ELA2 (Accession number NM_{—}001972):

| | |
|---|---|
| sense- | |
| 5′AATCCACGGAATTGCCTCCTTCGT-3′, | (SEQ ID NO.: 9) |
| antisense- | |
| 5′TTGTCCTCGGAGCGTTGGATGATA3′; | (SEQ ID NO.: 10) |

For GAPDH, a house keeping gene, used as an internal control for rq-PCR (Accession number NM_{—}002046):

| | |
|---|---|
| sense- | |
| 5′TTCGACAGTCAGCCGCATCTTCTT3′, | (SEQ ID NO.: 11) |
| antisense- | |
| 5′GCCCAATACCGACCAAATCCGTTGA. | (SEQ ID NO.: 12) |

The annealing and extension temperatures were 55° C. and 75° C. respectively, and lasted 20 second and 40 second respectively. All reactions were performed in triplicate for the standard curve and in quadruplicate for the experimental and the negative control. Rotorgene v4.6 and v5 software was applied for quantification and data analysis. The relative expression levels of ELA2 in the tests and controls were calculated as ratios to the expression levels of GAPDH.

### Immunoblotting and Immunocytochemistry

Western blot was performed as follows. Briefly, the HL60 or differentiated HL60 cells were collected 84 hours after siRNA transfections. The cells were lysed, and the lysates subjected to immunoblot analysis with a polyclonal antibody against ELA2 (Abcam, Cambridge, Mass.) raised in rabbits against full length protein, at a concentration of 50 ug/10 ml. The same blots were stripped and probed with an antibody against GAPDH to indicate the loading controls of the blots (Santa Cruz Biotechnology, Santa Cruz, Calif.). The immunoreactive proteins were detected using enhanced chemiluminescence reagents (Amersham, Piscataway, N.J.) and a LumiImager (FUJIFILM Medical Systems USA Inc., Stamford, Conn.). For immunocytochemistry, after cells were perfused on surfaces of targeting nanoparticles, the cells were collected and cultured for 6 h in a Lab-Tek™ II Chamber Slide™ System (Nunc, Tokyo, Japan) with growth medium, and then fixed, permeabilized and probed with or without a mouse monoclonal FITC-anti-P-selectin antibody against human P-selectin (BD Pharmingen) using 100 ul for 10⁶ cells.

### Neutrophil Elastase Activity Assay

HL60 cells were perfused through tubes coated with ELA2-siRNA-PS-DSPE-PEG-nanoparticles or control-siRNA-PS-DSPE-PEG nanoparticles, and the adherent cells were collected and cultured for 84 hours. The cells were harvested, washed and lysed in Triton X-100 lysis buffer containing protease inhibitor cocktail (Sigma; St. Louis, Mo.). Supernatants were obtained after centrifugation at 20,000×g in a microcentrifuge for 15 min at 4° C. Elastase activity was assayed using the chromogenic, neutrophil elastase-specific substrate N-sismethoxysuccinyl Ala-Ala-Pro-Val p-nitroanilide (Sigma, St. Louis, Mo.) that was reconstituted to a stock solution at a concentration of 15 mM in DMSO. The reaction volume of 130 μL contained 50 μg of cell lysate supernatant proteins, and 200 μM of substrate in Hank's buffered salt solution (Sigma, St. Louis, Mo.). Optical density measurements at 405 nm were taken at 5, 10, 20, 30, 40 and 50 min using a Smartspec plus Spectrophotometer (Bio-Rad, Hercules, Calif.).

### Statistical Analysis

Results were expressed as mean±SD. Statistical differences between the control and different time points were determined by using one-way analysis of variance and t-test. P-values <0.05 were considered significant.

### Results

### Building Target Specific Nanoparticles

SiRNA was condensed with protamine and double stranded calf thymus DNA to enhance its delivery efficiency. The encapsulation of siRNA was achieved by rehydration of a lipid thin film in the presence of condensed siRNA (FIG. 1b). The siRNA encapsulation efficacies were determined with RiboGreen assay. The intensities of RiboGreen fluorescence were measured with or without Triton X-100, showing a ˜90% encapsulation efficacy. To construct P-selectin target specific nanoparticles (PS-DSPE-PEG NP), sulfhydryl (—SH) groups were introduced into P-selectin by Traut's Reagent, and then the —SH groups of P-selectin were covalently linked to maleimide groups of DSPE-PEG2000 to form a PS-DSPE-PEG complex (FIG. 1a). An insertion step then conjugated PS-DSPE-PEG onto unilamellar liposomes to make targeted nanoparticles (PS-DEPE-PEG NP). To make non targeted nanoparticles (DEPE-PEG NP), DSPE-PEGs were inserted onto unilamellar liposomes without P-selectin (FIG. 1b). The specific targeting activity of nanoparticles was granted by P-selectin, whereas neutralization of the nanoparticle surface and stabilization of the particles was achieved by DSPE-PEG2000. Both effects were important for subsequent microtube coating and perfusion experiments. The target specificity of the nanoparticles was provided by the covalently attached P-selectin, an important endothelial cell adhesion molecule, onto the surface of the particles.

The physical characteristics of the nanoparticles are summarized in Table 1. The particle diameters of Cy3-siRNA Liposome and DSPE-PEG modified non-targeting nanoparticles were similar, whereas PS-DSPE-PEG modified targeting nanoparticles were 35 to 40 nm larger than non-targeting nanoparticles. The zeta potential was dramatically reduced by DSPE-PEG surface modification compared to those of naked nanoparticles. Attachment of PS-DSPE-PEG onto nanoparticles showed a slight increase of zeta potential compared to that of the nanoparticles modified only by DSPE-PEG, but remained lower than the naked nanoparticle zeta potential. It is likely that the sign and magnitude of the zeta potential could be controlled by varying the ratio of PEG and P-selectin on the liposomal surface.

TABLE 1
Characterization of Nanoparticles

| | Cy3-siRNA | DSPE- | PS-DSPE- |
|---|---|---|---|
| | Liposome | PEG NP | PEG NP |
| Particle size (nm) | 107.6 ± 11.6 | 110.6 ± 12.8 | 148.6 ± 18.6 |
| Zeta potential (mv) | 21.2 ± 4.64 | −5.4 ± 6.84 | 3.48 ± 5.3.6 |

Data are mean ± SD of four independent measurements

FIG. 1c contains images of siRNA liposomes taken with scanning electron microscopy, and shows that the liposomes were well formed by rehydration of lipid thin film with a mixture of siRNA, protamine and thymus double strand DNA. The size of the liposomes was in the range of 100 to 150 nm. This indicates that the results from scanning electron microcopy agree well with the liposome dimensions measured by dynamic light scattering (Table 1). To determine the efficiency of siRNA encapsulation by the liposomes, RiboGreen assay was used to measure the intensities of siRNAs in the presence or absence of Triton X-100, showing about 90% of total siRNA was encapsulated by the liposomes (FIG. 1d).

P-Selectin is Necessary for PS-DSPE-PEG Nanoparticle Absorption onto the Surface of Microtubing.

To test whether PS-DSPE-PEG nanoparticles could attach onto the surface of microtubing, the PS-DSPE-PEG nanoparticles were loaded into the microtube and incubated for 2 hours at room temperature (RT). After perfusion, nanoparticles without P-selectin modification were not retained in microtubes precoated with rhP/Fc (FIGS. 2a and b), whereas nanoparticles coated with P-selectin could coat onto the surface of the microtube and resist elution under shear stress (FIGS. 2c and d). These results indicate that P-selectin covalently attached onto the surface of nanoparticles was indispensable for absorption of the nanoparticles onto the surface of the microtube (FIG. 2).

### Cellular Uptake and Knockdown Efficiencies for Different Nanoparticles Under Static Conditions.

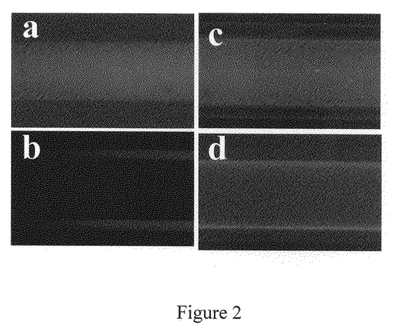

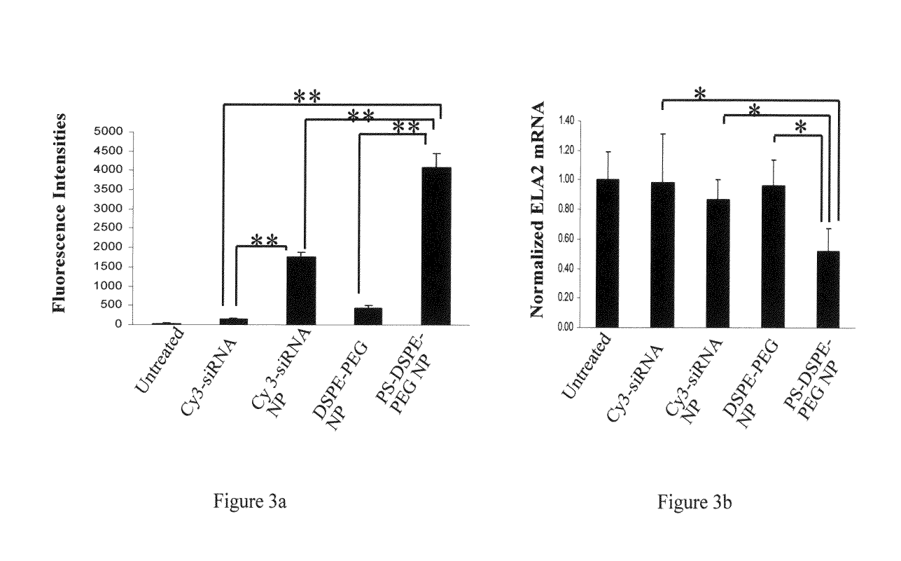

To investigate the delivery efficiency of different nanoparticles, we conducted a cellular uptake study using Cy3-labeled siRNA. The fluorescence intensities of the cell lysates were used to measure the siRNA cellular delivery efficiencies by different lipid vesicles. The lysate from cells treated with naked Cy3-siRNA showed a slightly higher fluorescence intensity than those from untreated cells, whereas the Cy3-siRNA encapsulated by liposomes (Cy3-siRNA Liposome) showed a significant (p<0.01) increase in fluorescence intensity (FIG. 3a). For comparison, attachment of DSPE-PEG-2000 maleimide to the particles (DSPE-PEG NP) reduced the siRNA delivery efficiency dramatically (FIG. 3a). Fluorescence intensity of cells treated with PS-DSPE-PEG-nanoparticles (PS-DSPE-PEG NP) showed 2.3-fold higher loading than cells treated with naked nanoparticles and 9.2-fold higher than cells treated with non-targeting nanoparticles (FIG. 3a), which indicates that P-selectin significantly (p<0.01) increased the Cy3-siRNA delivery efficacy to HL60 cells. The low siRNA delivery efficiency observed with PEGylated nanoparticles is most likely due to the PEGylation providing a steric hindrance for close contact between the cell and nanoparticle surfaces. The higher delivery efficiency for PS-DSPE-PEG nanoparticles might be the result of specific receptor-ligand interaction, which could facilitate the cellular internalization of the particles.

To test the relationship between siRNA uptake and gene knockdown efficiencies, siRNAs of neutrophil elastase (ELA2), a specific gene expressed in neutrophils, were delivered in naked or encapsulated form in different lipid vesicle formulations. The total RNA was extracted, and the resulting cDNAs from reverse transcription were used to perform real time quantitative-PCR (rq-PCR). Compared to control, no silencing was observed in the group of naked ELA2 siRNA. Furthermore, neither ELA2-siRNA encapsulated by Liposome or by DSPE-PEG nanoparticles showed significant knockdown of the ELA2 gene. However, ELA2-siRNA encapsulated within PS-DSPE-PEG nanoparticles showed significant (p<0.05) (50%) knockdown in ELA2 mRNA level compared to cells within the untreated group (FIG. 3b).

### PSGL-1-Mediated Uptake of PS-DSPE-PEG Nanoparticles.

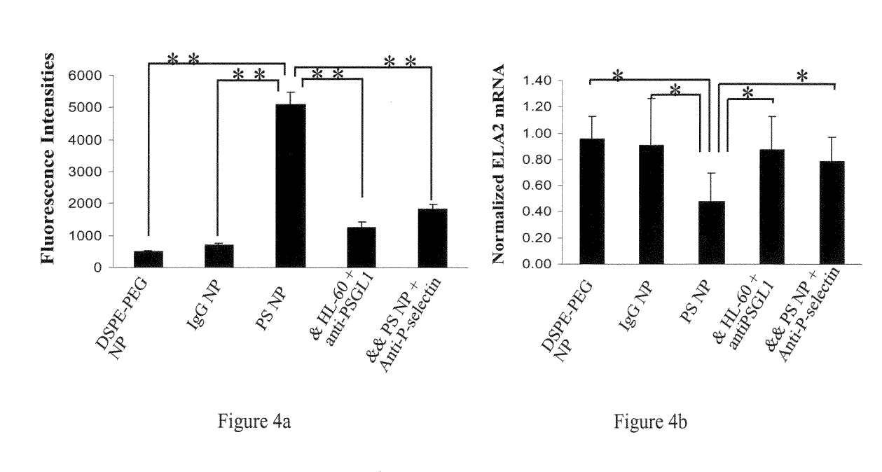

To assess the specificity of PS-DSPE-PEG nanoparticles, several strategies were applied to test the interaction of the particles and HL60 cells which are known to express PSGL-1. When nanoparticles were coated with IgG instead of P-selectin, uptake study showed that siRNA delivery efficiency was not notably higher than that of non-targeted nanoparticles, and significantly (P<0.01) lower than that of PS-DSPE-PEG nanoparticles. PSGL-1 receptors in HL60 cells were pre-blocked with anti-PSGL-1 antibody or PS-DSPE-PEG nanoparticles were pre-blocked with anti-P-selectin antibody, and then the uptake experiments repeated. Both showed a dramatic decrease of the uptake efficiencies compared to non-blocked samples (FIG. 4a). rq-PCR were used to confirm the specificity of PS-DSPE-PEG nanoparticles. No obvious ELA2 silencing was observed in the cells from IgG-attached nanoparticles, nor in cells pre-incubated with anti-PSGL-1 or from the PS-DSPE-PEG nanoparticles pre-incubated with anti-P-selectin in rq-PCR (FIG. 4b). However, HL60 cells transfected with PS-DSPE-PEG nanoparticles showed a dramatic knockdown in the mRNA level of ELA2 compared to those of the non-targeting nanoparticle group, IgG nanoparticle, HL60 pre-incubated with anti-PSGL-1 and PS-DSPE-PEG nanoparticle pre-incubated with anti-P-selectin groups in rq-PCR (FIG. 4b). These data support the notion that interaction of P-selectin and PSGL-1 is necessary for PS-DSPE-PEG nanoparticle binding and the delivery of siRNA into HL60 cells.

HL60 Cells Captured from Flow

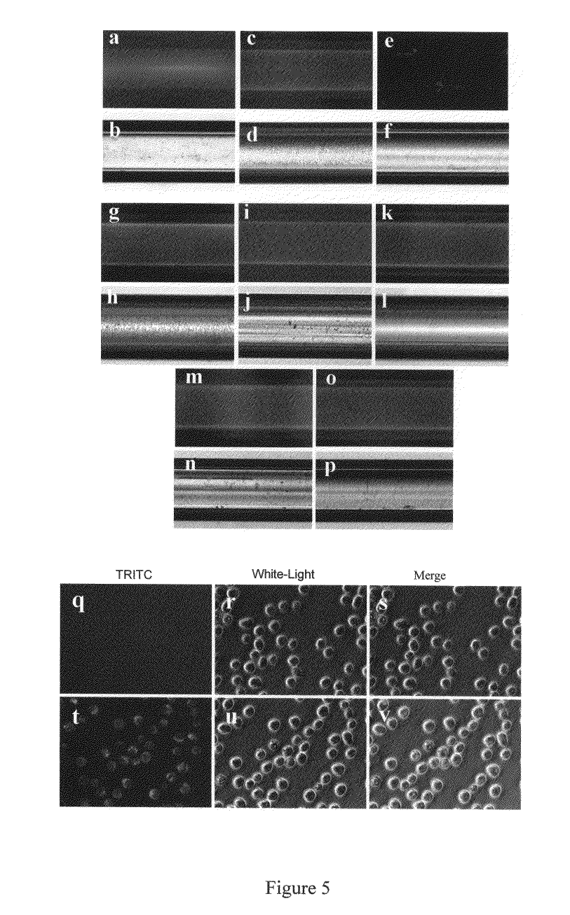

As shown in FIG. 2, PS-DSPE-PEG nanoparticles were effectively immobilized on the inner surface of microtubes. After coating with PS-DSPE-PEG nanoparticles, cellular perfusion experiments were performed with HL60 cells, a leukemic cell line known to express PSGL-1. After cellular perfusion, a near monolayer of HL60 cells was tethered (FIG. 5d) and accompanied by a thin layer of PS-DSPE-PEG nanoparticles absorbed onto the inner surface of the microtube (FIG. 5c). Furthermore, the coated PS-DSPE-PEG nanoparticles were released with the tethered cells by higher shear stress and air embolism from the microtube surface (FIGS. 5e and 5f). This indicates that the affinity of PS-DSPE-PEG nanoparticles to HL60 was greater than the nanoparticle affinity for the microtube surface.

To verify that PSGL-1 is necessary for the capture of HL60 cells by PS-DSPE-PEG nanoparticles under flow, we pre-incubated HL60 cells with anti-PSGL-1 for 1 hour at RT and then perfused these cells over the PS-DSPE-PEG nanoparticle surface. Alternatively, MCF7 cells, a breast cancer cell line which is known not interact with P-selectin, were perfused over the surface of PS-DSPE-PEG nanoparticle coated microtubes. Both of the perfusions showed negligible capture of cells onto the PS-DSPE-PEG nanoparticle surfaces (FIGS. 5j and 5l), which further confirm that PSGL-1 is indispensable for tethering and capturing the cells onto the PS-DSPE-PEG nanoparticle surface. The requirement of P-selectin for PS-DSPE-PEG nanoparticle surface capture of HL60 cells was demonstrated by using anti-P-selectin antibody to block P-selectin on PS-DSPE-PEG nanoparticles, and using a non-specific IgG to replace P-selectin to construct IgG nanoparticles. Both IgG nanoparticles and PS-DSPE-PEG nanoparticles blocked by anti-P-selectin antibody were shown to successfully coat onto the surface of microtubes (FIG. 51I g and i). However, neither the PS-DSPE-PEG nanoparticles that were pre-blocked by anti-P-selectin antibody nor the IgG-substituted particles had the ability to immobilize HL60 cells onto the coating surfaces (FIGS. 5n and 5p).

To measure the siRNA delivery efficiency of PS-DSPE-PEG nanoparticles under perfusion condition in microtubes, HL60 cells were infused into microtubes that were coated with Cy3-siRNA encapsulated in PS-DSPE-PEG nanoparticles or precoated with P-selectin and then Cy3-RNAs encapsulated by DSPE-PEG nanoparticles. Cy3-siRNA encapsulated by PS-DSPE-PEG nanoparticles were efficiently bound and delivered into HL60 cells (FIGS. 5t-v). The surface of microtubes pre-coated with P-selectin and then with Cy3-siRNA-DSPE-PEG nanoparticles could capture cells from flow (data not shown), but almost none of the Cy3-siRNA was delivered into the cells due to Cy3-siRNAs-encapsulated nanoparticles that were eluted out of the microtube during the perfusion (FIGS. 5q-s). In tubes coated with IgG nanoparticles, the absorption of the nanoparticles onto the surface of the microtube was quite effective, however the number of cells tethered onto the IgG nanoparticle surface was negligible (data not shown). To test the real-time uptake of the siRNA into the target cells by PS-nanoparticles, a time course study (1, 2, 4, 6 and 8 hours) for PS-nanoparticle delivery of siRNA into HL60 cells during rolling were conducted. The results showed that the peak in cellular uptake of PS-DSPE-PEG nanoparticle-Cy3-siRNA was reached after 2 hours of rolling (data not shown).

Taken together, these data suggest that the capture of HL60 cells by PS-DSPE-PEG nanoparticles is mediated by adhesion molecules rather than nonspecific binding, and the interaction of P-selectin with PSGL-1 is crucial for capturing and delivering siRNA into the specific target cells.

ELA2 siRNA-PS-DSPE-PEG Nanoparticles Silenced Neutrophil Elastase Under Perfusion Conditions in Microtube.

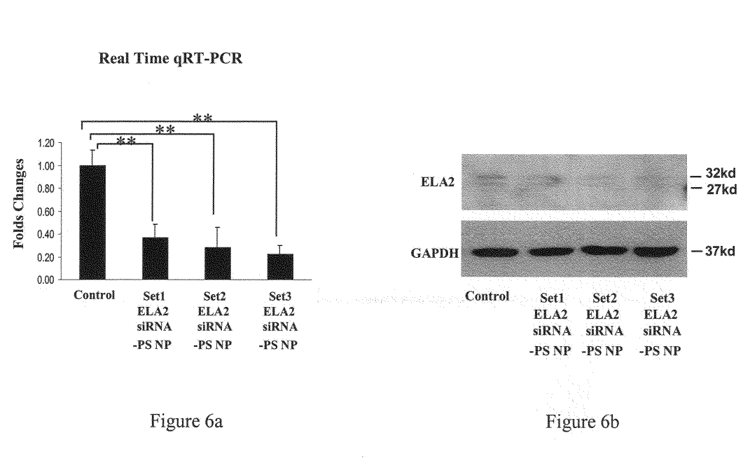

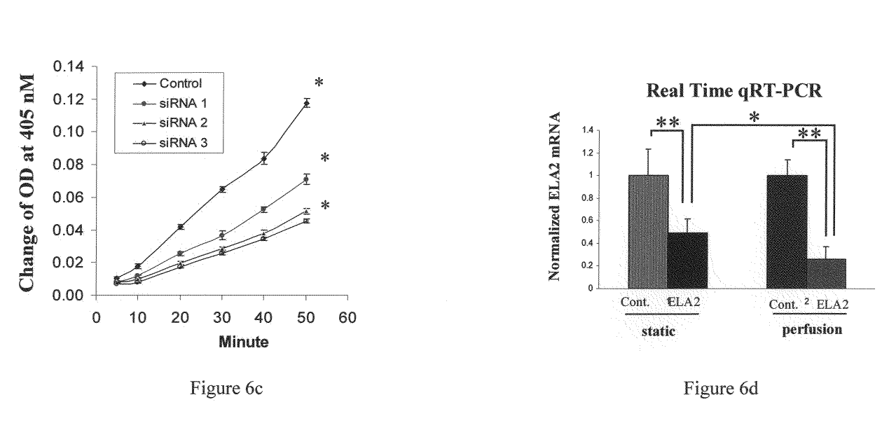

Three sets of ELA2 siRNAs were used for the study of ELA2 gene knockdown efficiency by ELA2 siRNA-PS-DSPE-PEG nanoparticles under perfusion conditions in microtubes. After cell perfusion, the adherent cells were collected by high shear stress combined with air embolism. The collected cells were cultured in 24 well dishes with 0.5 mL of growth medium. Total RNA was extracted and then reverse transcribed into cDNA for rq-PCR and after 48 hours of culture. Adherent HL60 cells were collected from the tube coated with control-siRNA encapsulated PS-DSPE-PEG nanoparticles as a control. Compared to the control, rq-PCR analysis showed significant (p<0.01) 63, 72 and 77% knockdown efficiencies by set 1, 2 and 3 of ELA2 siRNA, respectively in HL60 cells (FIG. 6a).

ELA2 knockdown by ELA2-siRNA-PS-DSPE-PEG-nanoparticles was also measured at the protein level by immunoblot analysis. After the cells were perfused over ELA2-siRNA-PS-DSPE-PEG-nanoparticles and cultured for 84 hours, all three sets of ELA2 siRNA showed substantial reduction of ELA2 gene translational levels (FIG. 6b, upper panel) compared to the control. The same blots were stripped and reprobed with an antibody against GAPDH, which verified the same amounts of the protein loaded into each lane of the Western blots (FIG. 6b lower panel). ELA2 activity assay was used to further confirm the knockdown of ELA2 by perfusion of cells on the surface of ELA2-siRNA-PS-DSPE-PEG-nanoparticles. As shown in FIG. 6c, OD reading showed a corresponding reduction of ELA2 activity in activity assays and protein level decrease in Western blot analysis.

To compare the siRNA knockdown efficacies between the static and perfusion conditions, total RNA was extracted from the cells transfected with ELA2 siRNA encapsulated PS-DSPE-PEG-nanoparticles under both conditions and reverse transcribed into cDNAs. The resulting cDNAs were used to conduct rq-PCR. Compared to controls, rq-PCR showed that mRNA levels of ELA2 were significantly (p<0.01) decreased by 51% and 74% under static and perfusion conditions, respectively (FIG. 6d). These data also indicate that rolling of cells over an ELA2-siRNA-PS-DSPE-PEG-nanoparticle surface resulted in significantly (p<0.05) higher mRNA knockdown efficiency compared to cells transfected under static conditions by ELA2-siRNA-PS-DSPE-PEG nanoparticles (FIG. 6d).

Delivery of siRNA into Granulocytes by PS-DSPE-PEG-Nanoparticle Surfaces Under Perfusion Conditions

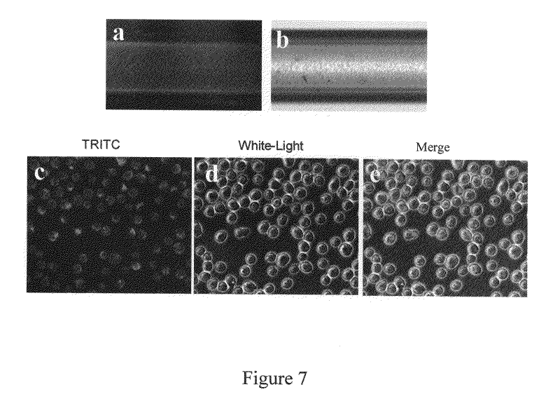

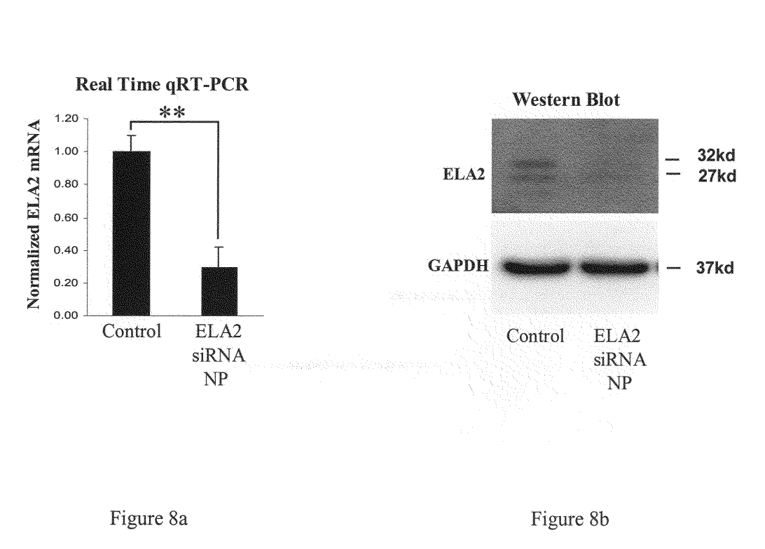

To test if granulocytes could be immobilized, siRNA delivered, and targeted gene silenced by a neutrophil elastase 2 siRNA PS-DSPE-PEG nanoparticle surface, differentiated HL60 cells were perfused over a surface of control Cy3-siRNA or neutrophil elastase 2 siRNA encapsulated PS-DSPE-PEG nanoparticles in microtubes. A near monolayer of granulocytes was captured by the PS-DSPE-PEG nanoparticle surface under flow (FIG. 7b). After perfusion, the adherent cells were collected and cultured. FIGS. 7c-e show that the delivery efficiency of Cy3-siRNA by PS-DSPE-PEG nanoparticles into granulocytes was as high as those of HL60 cells. The ELA2 knockdown efficiency was measured by rq-PCR and immunoblot. Compared to control, rq-PCR showed a significant (p <0.01) (71%) knockdown efficiency for ELA2 mRNA level in granulocytes (FIG. 8a). The images of Western blot showed a similar knockdown pattern as rq-PCR (FIG. 8b). The results of rq-PCR are normalized with GAPDH, and Western blot also featured GAPDH as loading control (FIG. 8b). These results indicate that the PS-DSPE-PEG nanoparticle coated surface has the ability to capture granulocytes from flow and deliver siRNA from the particles into the cells.

Using the method described herein, large numbers of cells were captured from flow by the PS-DSPE-PEG nanoparticle surface. Notably, the coated PS-DSPE-PEG nanoparticles were released with the collection of adherent cells, suggesting that the nanoparticles were tightly bound onto the surface of the cells (FIGS. 5a-f). By replacement of P-selectin with an IgG to modify the surface of the nanoparticles, the adhesive function of IgG-nanoparticles to the surface of the microtube was equal to that of PS-DSPE-PEG nanoparticles (FIGS. 5a-p). Therefore, the applications of the device can be broadened by antibodies directed against specific surface antigens. Blocking P-selectin by pre-incubation of PS-DSPE-PEG nanoparticles with anti-P-selectin, or PSGL-1 by pre-incubation of HL60 cells with anti-PSGL-1 nearly abolished the binding activity of HL60 cells with the PS-DSPE-PEG nanoparticle surface (FIGS. 5I, j, m and n), indicating that P-selectin on PS-DSPE-PEG nanoparticles and PSGL-1 on HL60 cells are necessary for the adhesion of HL60 cells. Using MCF7, a cell line without expression of P-selectin receptor, and IgG to replace P-selectin in the construction of nanoparticles, the cellular perfusion study confirmed that P-selectin on PS-DSPE-PEG nanoparticles and PSGL-1 on the circulating cells were both required for the cell rolling, tethering and capture by the PS-DSPE-PEG nanoparticle surface (FIG. 5k, l, o and p). Furthermore, cells failed to bind to the nonspecific IgG-nanoparticle surface, indicating that the binding of PS-DSPE-PEG nanoparticles to Fc receptors was minimal (FIG. 5p). Taken together, these results indicate that the cells captured from shear flow by the PS-DSPE-PEG nanoparticle coated device was due to the interaction of adhesion molecules with their cellular receptor, rather than due to nonspecific binding of the cells to lipids, Fc receptors, or the microrenathane surface.

Interestingly, a lack of correlation between vesicle uptake and mRNA knockdown efficiencies was observed among the different nanoparticles i.e., efficient uptake did not necessarily translate to efficient gene knockdown in the nontargeting particles. Target specific PS-DSPE-PEG nanoparticles provided 2.3-fold higher siRNA delivery efficiency than that of naked nanoparticles, and showed a 3.7-fold higher gene silencing activity than that of naked nanoparticles. While not intending to be bound by any particular theory, the explanation for this might be that target specific PS-DSPE-PEG nanoparticles deliver their encapsulated cargo through a receptor-mediated pathway, which not only has relatively higher affinity but also greater efficiency with its receptor recycling/trafficking mechanism. On the other hand, non-targeting naked nanoparticles deliver their cargo via a nonspecific charge:charge interaction.

Clearly the efficiency of the particle internalization through receptor-ligand interaction was greater than that for charge-charge. Another advantage of the device is that PS-DSPE-PEG nanoparticles were coated onto the surface of a microtube and would not be introduced into the circulation to distribute into organs and tissues, such as reticular connective tissue, lymph nodes, spleen and liver, thus partially avoiding the effect of the reticuloendothelial system. It is reasonable to expect that the immobilized PS-DSPE-PEG nanoparticles on the surface of microtube have relatively longer half-life than those in the circulation. Therefore, compared to particles introduced into the circulation, a lower dose of PS-DSPE-PEG nanoparticles in the device could be used to achieve the same effect of gene silencing.

The knockdown efficiency for cells perfused over the PS-DSPE-PEG nanoparticle surface was higher than that the equivalent static condition (FIG. 6d). When the cells were perfused over the PS-DSPE-PEG nanoparticle surface in the microtube, the interaction of the cells with PS-DSPE-PEG nanoparticles resulted in cell tethering, rolling and adhesion on the surface of PS-DSPE-PEG nanoparticles. In particular, when the cells were rolling on the surface of the particles, the whole cellular membrane has opportunity to contact and interact with the PS-DSPE-PEG nanoparticles. In this manner the rolling cells could pick up more PS-DSPE-PEG particles than cells resting static on the surface. Additionally, it is well known that shear stress tends to flatten rolling cells, thereby increasing the instantaneous contact area with an adhesive surface. Finally, the repeated microscale cell deformations which occur during rolling adhesion may enhance the intracellular transport and mixing of siRNA, although this remains to be directly demonstrated.

In this invention, we have developed a delivery device that can successfully capture targeted cells from physiological shear flow, and efficiently deliver payload molecules (such as siRNA) into targeted cells to achieve a desired result (such as silence a gene of interest). Most importantly, this device could be incorporated into the circulatory system in vivo and modify targeted cells in the bloodstream. This indicates that selectin-lipid hybrid (such as PS-DSPE-PEG) nanoparticle surfaces could capture cells from the blood stream and deliver contents of the particles into the captured cells. Thus, this device could be used for increasing the efficacy of therapeutic material delivery, reducing side effects and enhancing therapies directed at circulating cells in vivo.

### Example 2

### Delivery of siRNA to HL60 Cells Via a Solution-Based Delivery-Vehicle

In the previous Example, the P-selectin-receptor-specific-unilamellar nanoparticles were deposited on the lumen of a microtube prior to introduction of the HL60 targeted cell population, a situation that presents the decorating P-selectin on the nanoparticles optimally for interaction with the HL60 cells since it approximates the situation in a blood vessel. In a variation of the previous example, the compositions and methods of Example 1 can be used, except that the interaction of the nanoparticles with HL60 cells is allowed to occur in solution, i.e., the nanoparticles are not deposited on a surface.

The results can be analyzed using the same fluorescence assay as described in Example 1. The results can be expected to be similar to those of Example 1, i.e., delivery of Cy3-siRNA is also expected to occur when the experiments are performed in solution, rather than on a surface as was done in Example 1. In this situation it is expected that the nanoparticles will directly bind to cells to which they are targeted, so that siRNA will not be wasted on the majority of non-targeted cells.

### Example 3

### Delivery of siRNA to HSPC Cells

In this example, hematopoietic stem and progenitor cells (“HSPCs”) can be altered by introduction of siRNA such as the Cy3-siRNA used in the previous examples. The compositions and methods used to obtain this alteration are as for Examples 1-2, except that E-selectin can be used as the selectin in the hybrid selectin-lipid molecules constructed. The assays used to analyze the results obtained when these experiments are performed can also be as for Examples 1-2.

While specific embodiments of the present invention have been described in the foregoing, it will be appreciated by those skilled in the art that many equivalents, modifications, substitutions, and variations may be made thereto without departing from the spirit and scope of the invention.

What is claimed is:

1. A method for delivering a payload to a cell expressing a cell surface selectin ligand comprising:
a) providing liposomes, said liposomes comprising lipid molecules having selectin molecules covalently attached thereto and said liposomes having payload molecules encapsulated therein;
b) allowing the liposomes from a) to be immobilized to a flow surface;
c) allowing a fluid containing the cells expressing a cell surface selectin ligand to come in contact with the flow surface from b) under conditions permitting rolling of cells over said flow surface;
wherein rolling of cells results in delivery of the payload molecules to the cells.

2. The method of claim 1, where said cell expresses at least one P-selectin ligand.

3. The method of claim 2, where said at least one P-selectin ligand is P-selectin glycoprotein ligand-1.

4. The method of claim 1, where said liposomes comprises disteroyl phosphatidylethanolamine-polyethyleneglycol—P-selectin.

5. The method of claim 1, where said payload molecules are selected from the group consisting of DNA and RNA.

6. The method of claim 6, where the RNA is siRNA.

7. The method of claim 1, where said cell is a blood borne cell.

8. The method of claim 7, wherein said cell is a stem cell or a cancer cell.

9. The method of claim 8, wherein said cell is a blood cancer cell or metastatic cell.

10. The method of claim 1, wherein the liposomes are between 50 and 200 nm in diameter.

11. The method of claim 1, wherein the liposomes are unilamellar and/or multilamellar.

12. A device for delivery of payload molecules to cells comprising at least one microtube, wherein the inner surface of the microtube has liposomes attached thereto, said liposomes having payload molecules encapsulated therein and said liposomes having lipid molecules incorporated in the membrane, said lipid molecules having covalently bound selectin molecules.

13. The device of claim 12, wherein the lipid molecules having covalently bound selectin molecules are disteroyl phosphatidylethanolamine-polyethyleneglycol.

14. The device of claim 12, wherein the payload molecules are nucleic acids.

15. The device of claim 12, wherein the liposomes are unilamellar or multilamellar and are between 50 and 100 nm.

16. The device of claim 12, wherein the microtube has a diameter of 20-1000 microns.

17. The device of claim 12, wherein the device comprises a parallel array of microtubes, wherein microtube has a diameter of 20-1000 microns.

18. A composition comprising liposomes, said liposomes having lipid molecules incorporated in the membrane, said lipid molecules having selectin molecules covalently attached thereto.

19. The composition of claim 18, wherein said selectin is P-selectin, L-selectin, E-selectin, or a chimeric or mutant variant thereof.

20. The composition of claim 18, wherein the liposomes have payload molecules encapsulated therein.
